(19) United States
(12) Patent Application Publication
(10) Pub. No.: US 2014/0025163 A1
(43) Pub. Date: Jan. 23, 2014

(54) SYSTEMS, DEVICES AND METHODS FOR SURGICAL AND PRECUTANEOUS REPLACEMENT OF A VALVE

(71) Applicants: Saimuralidhar Padala, Atlanta, GA (US); Vinod H. Thourani, Atlanta, GA (US)

(72) Inventors: Saimuralidhar Padala, Atlanta, GA (US); Vinod H. Thourani, Atlanta, GA (US)

(73) Assignee: EMORY UNIVERSITY, ATLANTA, GA (US)

(21) Appl. No.: 13/978,912

(22) PCT Filed: Jan. 25, 2012
(86) PCT No.: PCT/US12/22533

§ 371 Date: Oct. 4, 2013

Related U.S. Application Data
(60) Provisional application No. 61/436,083, filed on Jan. 25, 2011.
(60) Provisional application No. 61/470,114, filed on Mar. 31, 2011.

(51) Int. Cl. A61F 2/24

(52) U.S. Cl. CPC A61F 2/2412; A61F 2/2418
USPC 623/2.18; 623/2.17

(57) ABSTRACT

The systems, devices and methods relate surgical and percutaneous replacement of a valve. The devices may include a frame and artificial leaflets. The devices may include one frame or two frames. The devices may also include a stent device in which the implant valve device may be implanted. The devices may also include a valve retainer device.

### CROSS REFERENCE TO RELATED APPLICATIONS

This application claims priority to Provisional Application Ser. No. 61/436,083, filed Jan. 25, 2011, and Provisional Application Ser. No. 61/470,114, filed Mar. 31, 2011, which are hereby incorporated by this reference in their entireties.

### FIELD

This disclosure relates to systems, devices and methods for surgical, minimally invasive and percutaneous replacement of heart valves. In some embodiments, the disclosure relates to a heart valve implant device having an outer structure of said material that is stiffer than the inner leaflets it supports in a desired shape. In other embodiments, the disclosure relates to multiple flexible elements that govern the motion of the flexible leaflets, and stents and stent systems in which a bioprosthetic valve, including the disclosed valve devices and systems, may be anchored.

### BACKGROUND

Heart valve disorders, for example, rank second in all cardiovascular diseases that contribute to mortality in the western and eastern worlds, and impose significant financial burden on healthcare systems. Currently, patients receive either medical treatment or surgical repair or replacement using open heart surgery. Medical treatment is only a medical management option that reduces the symptoms in these patients. However, medical treatment typically only delays the need for surgery. Most patients require surgery at some point in their life. Current standard of care for such diseased heart valves requires open heart surgery either for valve replacement or valve repair.

Valve replacement with mechanical valves is an issue as it requires lifelong anti-coagulation therapy, while bioprosthetic valves often fail within 10-15 years after implantation. Valve repair by reshaping the diseased tissue is becoming popular, but the durability of such repairs, has been very sub-optimal with persistent or recurrent valve regurgitation occurring in up to 64% of the patients. Further, open heart surgeries are extremely invasive and are risky to perform in older patients and those suffering from complications, such as diabetes, renal disease, or other associated complications. Both valve replacement and repair require cardiac arrest and placing the patient on cardiopulmonary bypass, and implantation of a ring or valve of a specific size determined by the surgeon in the operating room. Once the implantation is complete, assessment of the repair or replacement valve is performed on a flaccid heart, to assess acute success of the procedure, and then finally closing the heart, reviving it from its arrested state, and finally closing the muscle and chest incisions. Thus, the current valve replacement and repair procedures are invasive and are associated with risks.

Thus, there is a need for the development of transcatheter valve technologies that either reduce or completely eliminate the invasiveness of current valve replacement and repair procedures.

### SUMMARY

The disclosure relates to systems and devices to replace malfunctioning or diseased heart valves and methods to deliver and anchor these valve devices to the desired location using surgical or catheter techniques. In some embodiments, the disclosure relates to devices structured to replace the native valve. In some embodiments, the disclosure relates to stent devices that are structured to anchor a replacement valve device in the native valve. In other embodiments, the disclosure relates to implant systems.

In some embodiments, the device may include a frame structured to have a shape of the valve to be replaced and being structured for placement within the valve annulus. The device may also include at least one valve leaflet, the leaflet including artificial chordae tendineae that is structured to control the motion of the valve beyond the valve annulus. In some embodiments, the device may include more than one leaflet. The leaflets may be of the same size. The leaflets may be of different sizes.

In some embodiments, the frame may include a support base. The device may also include two or more struts that extend from the structure perpendicularly from a fulcrum, the struts being structured to have adjustable flexibility, and the struts being structured to be adjusted either before or after surgical implantation to alter the motion of the valve leaflets. The valve leaflet may include a material mounted on the frame, and the material is an artificial polymeric material or treated tissue.

In some embodiments, the frame may include a support structure. In some embodiments, the device may include two or more struts that extend perpendicularly from the frame. The device may also include a connector disposed on each strut. Each strut may include at least one tension member that extends from the support frame to the connector.

In some embodiments, the frame may include a tension device that is structured to adjust a length and a tension of the member. The length and the tension may be adjusted according to at least one of desired extension of leaflet motion and recoil force to retract the valve leaflet.

In some embodiments, the device may include a first frame and a second frame. The first frame may be structured to be placed within the valve annulus. The second frame may be structured to be fit into the first frame. The second frame may include the at least one valve leaflet. The second frame may include a support base and at least two struts that extend perpendicularly from the support base at a fulcrum, wherein the struts are structured to be flexible bend with the fulcrum. The second frame may include a connector structured to connect with the connector of the first frame. A connector may be disposed on each strut of the second frame. The connector of the second frame may include an eye. The connector of the first frame may be a hook. The first and second frame may further include adapters and fasteners, respectively. The adapters may be configured to receive the fasteners. The adapters may be hollow openings and the fasteners may be protruding members.

In some embodiments, the disclosure relates to a valve implant device configured to replace the native valve. The valve implant device may include an implant valve structured to have a shape of the native valve to be functionally replaced and being configured for placement within the valve annulus, the ring having at least one valve leaflet, the leaflet including artificial chordae tendineae that are structured to control the motion of the valve beyond the valve annulus. The artificial chordae may be flexible chords of a material whose proximal ends attach at one point and the distal ends that insert into various regions in the leaflets attached to the valve annulus. In further embodiments, the device may include more than one leaflet. In some embodiments, the leaflets may be the same size. In other embodiments, the leaflets are of different sizes.

In some embodiments, the implant valve may include a base support that may be an annular ring and may include two or more struts that extend from the annular ring perpendicularly from a fulcrum, wherein the two struts are structured to be flexible and bend with the fulcrum. In further embodiments, the implant valve may further include a material mounted on the base. In some embodiments, the material is at least one of an artificial polymeric material or treated tissue.

In other embodiments, the disclosure relates to a valve implant device for surgical replacement of a valve of a patient, the valve having a valve annulus, including an annular frame, the annular frame including an annular ring that is structured according to the size and shape of the valve annulus, and two or more struts that extend perpendicularly from the annular ring, wherein each arm includes tension member having an end connected to a connector.

In some embodiments, the implant device may include a frame including an annular ring and two or more struts that extend from the annular ring perpendicularly from a fulcrum, wherein the two struts are structured to be flexible and bend with the fulcrum. In further embodiments, the struts may each include a connector structured to connect to another frame and lock the frame in place. In other embodiments, the tension member may be structured to determine motion of the struts of the implant valve, to tether the metallic chordae, and to determine the motion of the valve leaflets towards an annular plane of the valve and thereby their curvature and coaptation.

In other embodiments, the tension member may be connected to tension device that is structured to adjust a length and a tension of the tension member. In further embodiments, the length and the tension may be adjusted according to at least one of desired extension of leaflet motion and recoil force to retract the valve leaflets. In other embodiments, the length and the tension may be adjusted upon implantation of the valve on a functioning organ, such as a beating heart, using a catheter system. In further embodiments, the device may be structured to be implanted into a collapsible stent that is structured to be delivered to the valve for implantation using at least one of a catheter or surgically. In some embodiments, the device may further include an adjustable skirt structured to be adjusted to eliminate paravalvular leakage around the valve after implantation. In other embodiments, the device may be structured for suture, suture-less or percutaneous procedures for at least one of an adult and a pediatric patient. In some embodiments, the valve may be a heart valve. In other embodiments, the valve may be a venous valve or a gastric valve.

In other embodiments, the disclosure relates to a valve device for surgical replacement of a valve of a patient. The valve device may include: an annular ring; artificial valve leaflets that are sutured onto the annular ring; and at least two commissural struts, wherein struts are structured to control motion of the leaflets by deflection of the struts.

In further embodiments, the struts may have adjustable flexibility, and wherein the struts are structured to be adjusted either before or after surgical implantation to alter the motion of the valve leaflets. In some embodiments, the valve may be a heart valve. In other embodiments, the valve may be a venous valve or a gastric valve.

In other embodiments, the disclosure relates to a surgical replacement valve device for a valve of a patient. The device may include a frame having at least one elastic protrusion protruding therefrom, the protrusion having a connection mechanism; and an inner valve having an annulus ring and at least one strut protruding therefrom, the strut having a connection mechanism complementary to the connection mechanism of the frame. In further embodiments, the inner valve may include metallic chordae tendineae structured to be inserted into the valve. In other embodiments, the device may include a stent structured to support the insertion of the frame and inner valve into the native valve. In some embodiments, the valve may be a heart valve. In other embodiments, the valve may be a venous valve or a gastric valve.

In other embodiments, the disclosure relates to a valve device for surgical replacement of a valve of a patient. The valve device may include: an annular ring; a flexible wire attached to the annular ring; and at least two flexible elements extending between the annular ring and the flexible wire, the at least two flexible elements being structured to have a predetermined curvature range. In further embodiments, each of the flexible elements may include either a predetermined pattern on one side or may be made of composite materials of two or more different materials. The pattern or the composite materials may be configured to limit a range of curvature of each flexible element. In other embodiments, the flexible elements may include at least one protrusion. In further embodiments, the valve device may include a material that covers the flexible elements to form at least two leaflets. In further embodiments, the valve device may include a first flexible wire and a second flexible wire. In some embodiments, an area between the first flexible wire and the second flexible wire may be structured to be a surface onto which the leaflets coapt.

In other embodiments, the disclosure relates to a device for implantation into a valve of a patient. The device may include: an annular ring; at least two struts; and at least two leaflets, each leaflet including at least one flexible element that is structured to restrict motion of each leaflet. In further embodiments, the valve device may further include a flexible wire, the at least two leaflets extending between the annular ring and the flexible wire, the flexible wire being structured to be a surface onto which the at least two leaflets coapt.

In some embodiments, the disclosure relates to a stent device structured for use with a valve implant device for replacement of a heart valve of a patient, the valve having native valve leaflets and orifice. The stent device may include at least two segments. The segments may be structured to receive a valve implant device.

In some embodiments, the stent device may be structured to radially push the native valve leaflets outwards and rest on an annulus of the valve and during ventricular pressurization provide a radially inward force to hold the valve implant device in place.

In some embodiments, each segment may have a same shape and size. In other embodiments, each segment may have a different shape and size. In some embodiments, a segment may have a symmetric shape. In some embodiments, a segment may have a plurality of diameters.

In some embodiments, the stent may include at least two connectors, each connector connecting the two segments. The segments may be structured to pivot about the connector.

In some embodiments, each segment may include two openings, each opening on an opposing side. The openings may be configured to receive the connectors. In other embodiments, each segment may include a plurality of openings. In some embodiments, segment may be structured for a plurality of configurations.

In other embodiments, the disclosure relates to a stent device configured for use with a valve implant device for surgical replacement of a valve of a patient, the valve having native valve leaflets and orifice. In one embodiment of this stent may include two segments, the segments being configured to have a shape of the valve so that a radial force is applied on the native valve leaflets when the stent is implanted in the orifice, a passage is formed in the valve, the passage being configured to receive a valve implant device.

In some embodiments, the disclosure relates to an implant system for surgical replacement of a valve of a patient, the valve having a valve annulus, an orifice and native leaflets. The system may include an implant valve device structured to have a shape of the valve to be replaced and being structured for placement within the valve annulus, the device including at least one frame and at least one valve leaflet, the leaflet including artificial chordae tendineae that is structured to control the motion of the valve beyond the valve annulus; and a stent device, the stent device being structured to have a shape of the valve so that when a radial force is applied on the native valve leaflets when the stent is implanted in the orifice, a passage is formed in the valve, the passage being structured to receive the implant valve device when implanted into the orifice. In some embodiments, the stent device may include two movable segments. In some embodiments, the implant system may further include a valve retainer device structured to secure the implant valve device, the valve retainer device being structured to fixedly connect to the stent device.

In other embodiments, the system may include: an implant valve structured to have a shape of the valve to be replaced and being configured for placement within the valve annulus, the ring having at least one valve leaflet, the leaflet including artificial chordae tendineae that is structured to control the motion of the valve beyond the valve annulus; an annular frame, the annular frame including an annular ring that is structured according to the size and shape of the valve annulus, and two or more struts that extend perpendicularly from the annular ring, wherein each arm includes one elastic member having an end with a metallic protrusion; and a stent, the stent being structured to have a shape of the valve so that when a radial force is applied on the native valve leaflets when the stent is implanted in the orifice, a passage is formed in the valve, the passage being configured to receive the annular frame when it is implanted into the orifice. In some embodiments, the valve may be a heart valve. In other embodiments, the valve may be a venous valve or a gastric valve.

In some embodiments, the disclosures relates to a kit. The kit may include an implant device according to embodiments. The kit may include at least one frame. In some embodiments, the kit may include different flexible members. In some embodiments, the kit may include a stent device. In some embodiments, the kit may include a plurality of different segments. In some embodiments, the kit may include one, some, or all of an implant device, a stent and a valve retainer device. In some embodiments, the kit may include a valve implant system that may include a stent and an implant device.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows a surgical valve implant device for a valve according to embodiments;

FIG. 2 shows a frame of the valve implant device;

FIG. 3 shows a side view of the frame;

FIG. 4 shows another frame of the valve implant device according to embodiments;

FIG. 5 shows the tension device when a valve leaflet is in a closed position;

FIG. 6 shows the tension device when the valve leaflet is in an open position;

FIG. 7 shows a side view of a surgical valve implant device for a valve according to other embodiments;

FIG. 8 shows a top view of the surgical valve implant device;

FIG. 9 shows operation of the implant device implanted in a mitral valve in the diastolic open position;

FIG. 10 shows operation of the implant device implanted in the mitral valve in the systolic closed position;

FIG. 11 shows a surgical valve implant device according to other embodiments;

FIG. 12 shows a surgical valve implant device according to other embodiments;

FIGS. 13 (a)-(e) show different embodiments of a feature of the surgical valve implant device;

FIGS. 14 (a)-(d) show a cross-sectional view of the embodiments shown in FIGS. 13 (a)-(d), respectively;

FIGS. 15 (a)-(e) show a side view of the embodiments shown in FIGS. 13 (a)-(e), respectively;

FIG. 16 shows the surgical valve implant device implanted in the diastolic open position;

FIG. 17 shows the implant device implanted in the systolic closed position;

FIGS. 18(a)-(c) show a surgical valve implant device according to other embodiments;

FIGS. 19(a)-(c) shows a surgical valve implant device according to other embodiments;

FIGS. 20(a)-(c) show a surgical valve implant device according to other embodiments;

FIG. 21 shows a view of an implanted device;

FIG. 21 show a stent device according to embodiments;

FIG. 22 shows a segment of a stent device according to embodiments;

FIG. 23 shows a segment of a stent device according to embodiments;

FIG. 24 shows a segment of a stent device according to embodiments;

FIG. 25 shows a segment of a stent device according to embodiments;

FIGS. 26(a) and (b) show a segment of a stent device according to embodiments;

FIG. 27 shows a segment of a stent device according to embodiments;

FIG. 28 shows a stent device according to other embodiments;

FIGS. 29(a)-(c) show a stent device according to some embodiments;

FIG. 30 shows an example of a stent device according to embodiments;

FIG. 31 shows a valve retainer device according to embodiments; and

FIG. 32 shows a valve device implanted into a valve retainer device.

### DETAILED DESCRIPTION OF THE EMBODIMENTS

The following description, numerous specific details are set forth such as examples of specific components, devices, methods, etc., in order to provide an understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed devices, systems, and methods specifically address the patient variability in valve structure, function, and the need for devices that conform to the desired shape in individual patients at the time of surgery, acutely after surgery or several years after surgery. More particularly, the disclosed devices and methods specifically address the patient variability in heart valve structure, function and the need for beating heart adjustable devices that conform to the desired shape in individual patients at the time of surgery, acutely after surgery or several years after surgery.

The disclosed systems, devices and methods are described with respect to valves of a heart. However, it will be understood that the systems and devices, unless otherwise noted, may be used for replacement of other valves of a patient. As used herein, “patient” refers to any animal, preferably human, livestock, or a domestic pet. These valves may include, but are not limited to, venous valves and gastric valves.

It will be understood that the systems and devices disclosed, unless otherwise noted, may be implanted in patients undergoing first-time surgery; or these systems and devices can be implanted in patients who had a failure of a previous repair. Also, unless otherwise noted, the systems and devices disclosed may be implanted using surgical, minimally invasive or percutaneous techniques. The devices may be also used in traditional invasive procedures, such as open-heart surgical techniques. These devices may be structured to be implanted into the valve structure under image guidance and repositionable during implantation. Although the valve system shown in the Figures relates to the mitral valve, it will be understood that it is within the one of ordinary skill to modify the valve system for other heart valves, as well as other valves of the body.

### Valve Implant Devices

In some embodiments, the disclosure relates to valve implant devices structured for valve replacement. In some embodiments, the valve implant devices may be structured to be implanted directly within the valve. In other embodiments, the valve implant device may be structured to be used with a system for sutureless implanting. For example, the valve implant devices may be used with the system disclosed.

In some embodiments, the valve implant device may include more than one frame. In some embodiments, the valve implant device may include a first frame and a second frame that engages with the first frame. The first frame may be an outer frame, and the second frame may be an inner frame. FIG. 6 show an example of a valve device according to these embodiments.

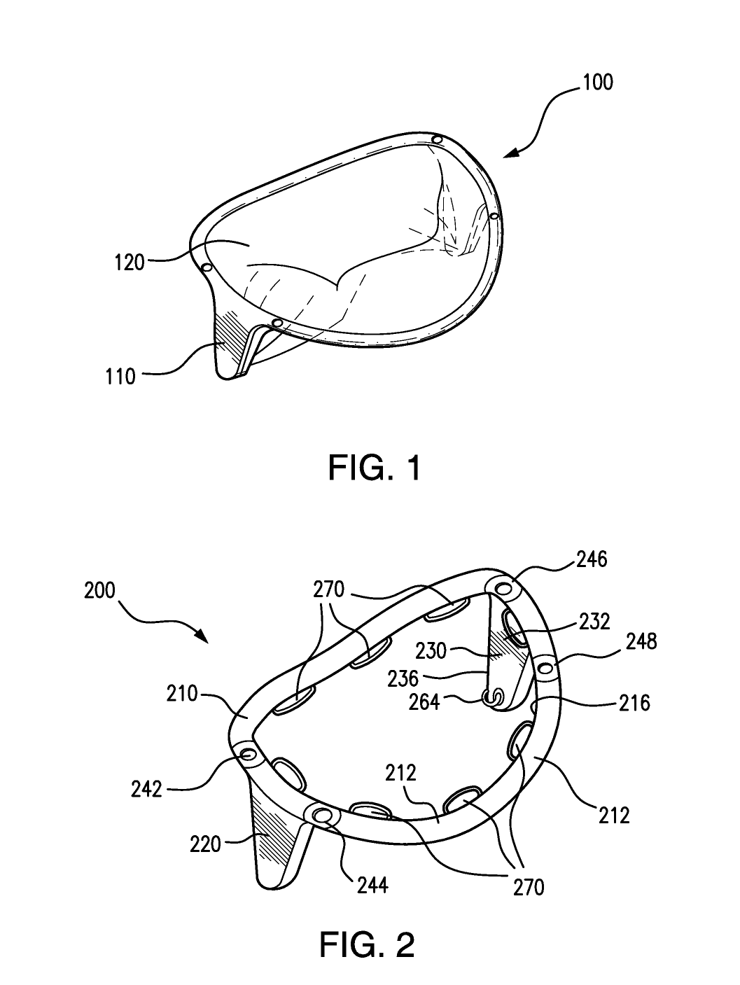

In some embodiments, the first frame may be a support structure structured to receive the second frame. The second frame may include an artificial valve. FIG. 1 shows a valve implant device 100 according to these embodiments. The valve implant device 100 may include a first frame 110 and a second frame 120.

In some embodiments, the first frame may include a support base frame (also referred to as “base”). The base may be an annular ring. The base may be shaped according to the anatomy of the heart valve. As shown in the Figures, the base may generally have a D-shape to correspond to the anatomy and shape of a mitral valve. FIG. 2 shows an example of a frame 200 that includes a base 210. However, the base is not limited to this shape and may have a different shape depending on the valve and its anatomy. For example, the base may have a round or circular shape, an oval shape, a C-shape, a U-shape, an open circle shape, and other curvilinear shapes. The base may also be structured to be adjusted to the desired shape upon implantation into the heart.

The base may be made of a metal material. The base may be made of a memory alloy that can conform to the desired shape upon implantation into the heart. The alloy may be, but is not limited to, Nitinol (also known as nickel titanium). In other embodiments, the base may be made of a biocompatible polymer.

In some embodiments, the base may be hollow. The base may be hollow along its entire circumference or a part of its circumference.

In some embodiments, the first frame may include at least one strut. Each of the struts may extend perpendicular from the base. Each strut may be structured to extend into the cardiac tissue, e.g., the ventricle, of a patient's heart when positioned into the patient's heart. The strut may be a commissure strut or post. The strut may extend from the base at a fulcrum so that the strut may be capable of bending.

The first frame may include any number of struts. The number of struts may depend on the heart valve that requires replacement. The number of struts may be equivalent to the number of valve leaflets determined to be mounted onto the second frame (the metallic annulus). For example, if the heart valve is the mitral valve, there may be two struts. If the heart valve is the tricuspid valve, there may be three struts.

In some embodiments, the strut(s) may be of the same material as the base. The strut may be of a different material. In some embodiments, the struts may be made of a biocompatible material that is capable of tensing and releasing. The material may be any material that has the desired elasticity. The struts may be integral with the base. In other embodiments, the strut(s) and the base may be separate components. The strut(s) and base may be fixedly disposed by a fastener. In some embodiments, the strut may be hollow or partially hollow. In some embodiments, as shown in FIG. 2, the first frame 200 may include two struts 220 and 230, which correspond to the anatomy of a mitral valve.

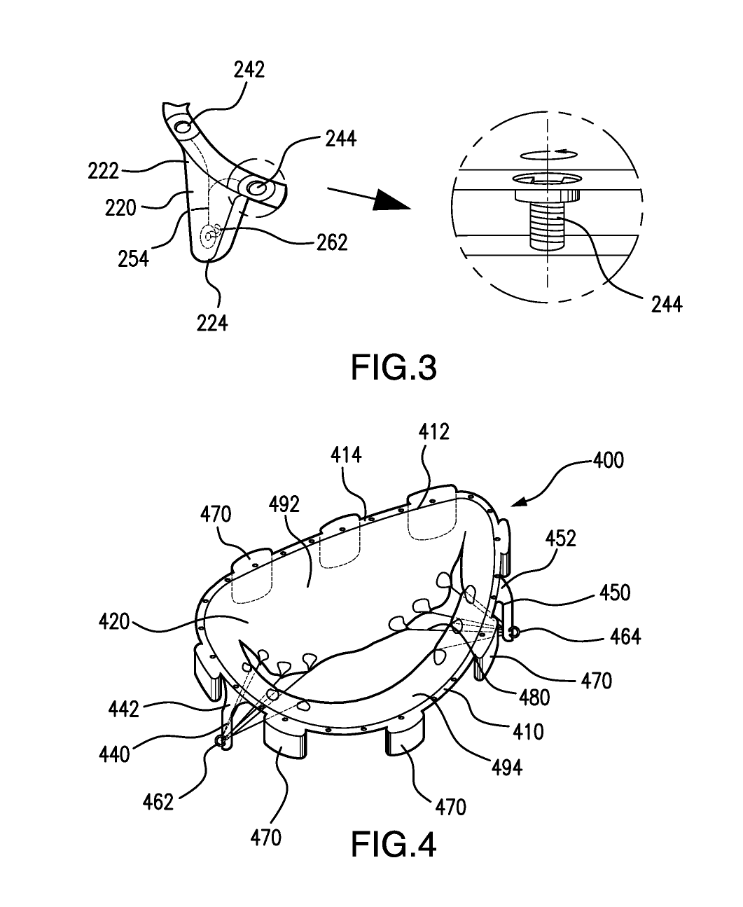

In some embodiments, the first frame may include at least one connector. The connector may be structured to connect to the second frame so as to adjust the tension of the artificial leaflets. The first frame may include any number of connectors. In some embodiments, the first frame may include a connector for each strut. In other embodiments, the first frame may include more than one connector for each strut. The connector may be disposed on each strut close to a second end (the end opposing the base). The connector may be disposed on the inner surface of each strut. The connector may be made of the same or different material as the strut. The connector may be made of an elastic material, such as an elastic band. In some embodiments, the connector may be a hook. As shown in FIGS. 2 and 3, the struts 220 and 230 may include connectors 262 and 264, respectively. As shown in FIG. 2, the strut 230 may be disposed on inner surface 236 of the strut 230.

In some embodiments, the first frame may include a tension device. The tension device may be structured to control the tension within the strut. In some embodiments, a tension device may control one, some, or all of the strut(s). The first frame may include any number of tension devices. In some embodiments, the first frame may include a tension device for each strut. In other embodiments, the first frame may include two tension devices for each strut. In some embodiments, the tension device may be a screw.

As shown in FIGS. 2 and 3, the first frame 200 may include tension devices 242, 244, 246, and 248. The tension devices 242 and 244 may be structured to control the tension of the strut 220 and the tension devices 246 and 248 may be structured to control the tension of the strut 230.

The tension device may be disposed on the base and may communicate with a tension member. The tension member may be disposed within or along the strut(s). In some embodiments, the tension member may include an elastic material capable of tensing and releasing. The tension member may be any material having the desired elasticity, for example, an elastic band. The tension member may extend from the tension device along most of the length of the strut. The tension member may be connected to the connector(s) of each strut. In some embodiments, there may be any number of tension members. In some embodiments, the tension member may correspond to the number of struts and/or tension devices. For example, the first frame may include one tension member for each strut that communicates with each tension device.

In some embodiments, the tension device may be structured to adjust the position and tension of the strut by increasing and/or decreasing the tension of the tension member. In some embodiments, the tension device may be accessible on the top surface 212 of the base 210. In some embodiments, the tension device may be structured to be rotated by a rotation device so as to adjust the tension of the tension member. In some embodiments, the tension device may be a head of a screw and the rotation device may be a complimentary screw driver. In some embodiments, the tension of the strut may be increased by increasing the tension of the tension member, i.e., shortening the length of the tension member. The tension of the strut may also be decreased (i.e., released) by decreasing the tension of the tension member, i.e., increasing the length of the tension member. The tension of the tension member may be structured to adjust the position of the connector. For example, as the tension device increases tension, the connector may be tensioned against inward movement; and as the tension member releases tension, the connector may be capable of moving back towards its original (non-tensioned) position. This movement adjusts the leaflets so as to adjust the position of the artificial valve according to the embodiments. When the connector is pulled, it may move and may then be elastically pulled back to open the valve.

FIG. 3 shows an enlarged view of one of the struts. As shown in FIG. 3, the strut 220 may include a tension member 254. The tension member 254 may extend from a first end 222 toward a second end 224 of the strut 220. The tension member 254 may extend to the connector 262. The tension member 254 may communicate with or connect to the connector 262. The tension member 254 may communicate with each of the tension devices 242 and 244. An enlarged view of one of the tension devices, the tension device 244, is shown in FIG. 3. As shown in FIG. 3, the tension may be adjusted by rotating the tension device 244. Although not shown, the strut 230 may include a tension member that communicates with tension devices 246 and 248.

The base may further include additional structure to improve the anchoring of the first frame onto the valve. For example, the ring may further include skirt elements or hooks. These elements maybe made of a metal or shape alloy, such as Nitinol.

In some embodiments, the first frame may further include at least one adapter. The adapter may be structured to receive complementary members of the second frame so as to fixedly dispose the second frame onto the first frame. In some embodiments, the first frame may include any number of adapters. In some embodiments, a plurality of adapters may be disposed along the inner circumference of the base. The adapters may be spaced out evenly or unevenly along the inner circumference of the base. The adapters may be any fastener or connector complimentary to the corresponding members of the second frame. In some embodiments, the adapters may have the same shape, different shapes, or a combination thereof. The adapters may be hollow rings that protrude from the inner circumference. As shown in FIG. 2, the frame 200 may include a plurality of adapters 270 disposed along inner circumference 216 of the base 210.

In some embodiments, the second frame may be an artificial valve device. The second frame may include an artificial leaflet(s) attached or mounted to a support base (also referred to as a valve skeleton and support frame). In some embodiments, the artificial leaflets may be made of pericardial tissue, native valve tissue, a polymer, or a combination thereof.

In some embodiments, the base may be an annular ring. The base may be shaped according to the anatomy of the heart valve. The base may have the same shape as the base of the first frame. The base may be of a different size. As shown in the Figures, the base may generally have a D-shape to correspond to the anatomy and shape of a mitral valve. However, the second base is not limited to this shape and may have a different shape depending on the valve and its anatomy. For example, the base may have a round or circular shape, an oval shape, a C-shape, a U-shape, an open circle shape, and other curvilinear shapes. The base may also be structured to be adjusted to the desired shape upon implantation into the heart.

The base may be made of a metal material. The base may be made of a memory alloy that can conform to the desired shape upon implantation into the heart. The alloy may be, but is not limited to, Nitinol (also known as nickel titanium). In other embodiments, the base may be made of a biocompatible polymer.

FIG. 4 shows an example of a second frame 400. As shown in FIG. 4, the second frame may include a base 410 and an artificial valve 420. The artificial valve may include more than one leaflet. The number of leaflets may depend on the valve to be replaced. For example, the artificial valve may include two leaflets for a mitral valve, three leaflets for a tricuspid valve, etc. The artificial valve may be mounted on the base 410 so as to extend from inner surface 412 of the base 410. The frame 400 may also include two leaflets, leaflet 492 and leaflet 494.

The second frame may include at least one fastener structured to fixedly dispose the second frame to the first frame. The fasteners may be disposed along a bottom surface of the base. The fastener(s) may be complimentary to the adapter(s) of the first frame. For example, the position, shape, and number of fastener(s) may be complimentary to the position, shape, and number of adapter(s) of the first frame. In some embodiments, the adapter may be a protrusion that has a shape that corresponds to the hollow ring of the first frame. For example, as shown in FIG. 4, the second frame 400 may include a plurality of fasteners 470 disposed on outer surface 414 of the base 410.

In some embodiments, the second frame may include at least one strut. Each strut may extend from a bottom surface of the base. Each strut may be structured to extend from an annulus into a cardiac chamber, e.g., the ventricle, of a patient's heart when positioned into the patient's heart. The strut may be a commissure strut or post.

The second frame may include any number of struts. The number of struts may depend on the heart valve that requires replacement. The number of struts may be equivalent to the number of valve leaflets determined to be mounted onto the first frame. For example, if the heart valve is the mitral valve, there may be two struts. If the heart valve is the tricuspid valve, there may be three struts. The number may be equivalent to the number of valve leaflets determined to be mounted onto the second frame. The number may also be equivalent to the number of struts disposed on the first frame. In some embodiments, the strut(s) may be of the same material as the base. The strut may be of a different material. In some embodiments, the struts may be made of a biocompatible material that is capable of tensing and releasing. The material may be any material that has the desired elasticity. The struts may be integral with the base. In other embodiments, the strut(s) and the base may be separate components.

In some embodiments, as shown in FIG. 4, the frame 400 may include two struts 440 and 450, which corresponds to the anatomy of a mitral valve. These struts may extend from the top surface of the valve into the ventricle of a patient's heart when positioned into the patient's heart.

In some embodiments, the second frame may include connectors. The connectors may be disposed on the inner surface of each strut. The connectors may be complementary to the connectors of the first frame so as to connect the struts of the first frame and second frame. In some embodiments, the complementary connectors may be an eye.

As shown in FIG. 4, the second frame 400 may include a connector 462 disposed on the strut 440. The connector 462 may be disposed on an outer surface 442 on the strut 440. The second frame may also include a connector 464 disposed on the strut 450. The connector 464 may be disposed on an outer surface 452 of the strut 450.

The second frame may include chordae tendineae. The second frame may include plurality of chordae tendinae. In some embodiments, the chordae tendinae may be made of a biocompatible metal. In some embodiments, the chordae tendineae may be disposed on each leaflet. In some embodiments, thee chordae tendineae may be embedded into all or part of the surface of each leaflet of the artificial valve. In other embodiments, the chordae tendineae may be disposed on all or part of the surface of each valve leaflet. In some embodiments, the chordae tendinae may be attached to the leaflets.

The chordae tendinae may extend from each leaflet to a commissural strut. The chordae tendinae may extend to a side of the strut opposing the connector. The chordae tendinae may terminate one the opposing surface. The number of chordae tendinae may be any number. The number for each leaflet may be the same or different. The chordae tendinae may be structured to control the basal motion of each of the valve leaflets.

As shown in FIG. 4, the frame 400 may include a plurality of chordae tendinae 480 disposed on each leaflet 492 and 494. A plurality of tendineae 480 may extend from the strut 440 to each leaflet 492 and 494. In addition, a plurality of tendineae 480 may extend from the strut 450 to each leaflet 492 and 494.

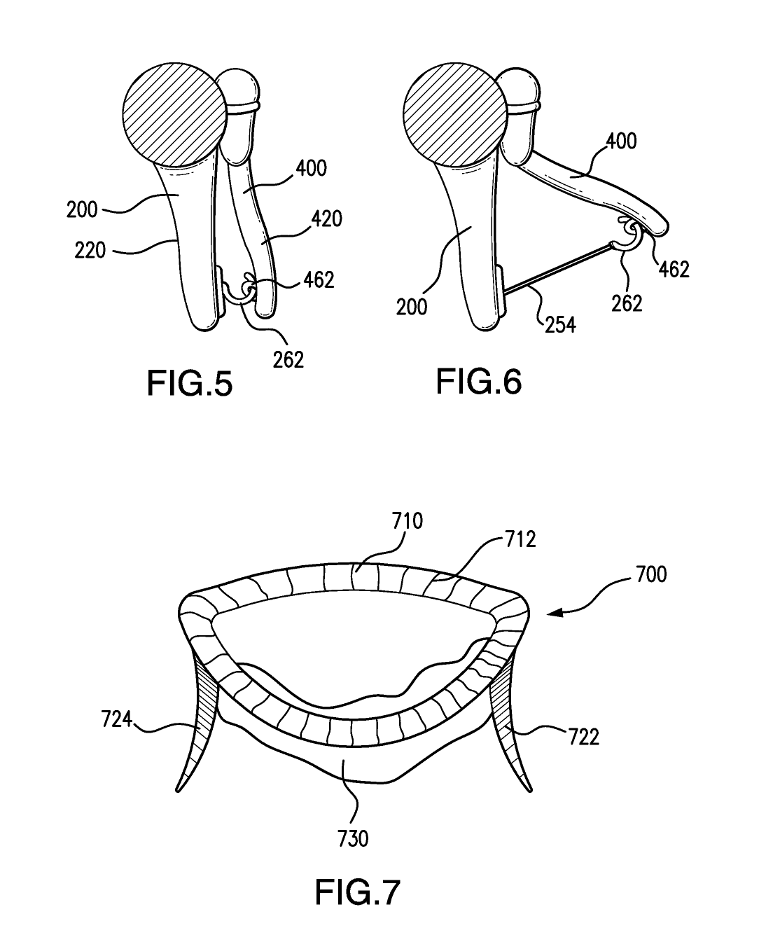

FIGS. 5 and 6 shows an example of how the first frame may lock and cooperate with the second frame. FIG. 5 shows an example of the valve device in an open position and FIG. 6 shows an example of the valve device in a closed position. As shown in FIG. 5, the connector 462 (disposed on the strut 440) of the second frame 400 may cooperate with the connector 262 (disposed on the strut 220) of the first frame 262. The connector 462 may latch onto the connector 262. As shown in FIGS. 5 and 6, this configuration may allow the connectors to pull on the tension device embedded into the frame 200 when the leaflets move basally. Specifically, when pressure across the bioprosthetic valve leaflets increases, the leaflets may move upward tethering on the chordae tendineae which in turn causes the connectors of the second frame to pull on the connectors of the first frame. The connectors of the second frame may then extend out of the frame to the extent that the strut of the first frame may extend and then the connectors of the first frame may be elastically pulled backwards by the tension member. The leaflets may thus move basally only as much as the tension member is structured to extend (as noted above, the extensibility of the tension member may be adjusted as required). The valve leaflet motion may thus be controlled without relying on the pressure of the heart.

The leaflet shapes may be predetermined such that optimal closure of the leaflets may be achieved, for example, when a transvalvular pressure gradient is applied. The commissural struts may thus be capable of restricting the leaflets from abnormally prolapsing above their annulus, by restricting distance/arc length the struts/protrusions can traverse from their perpendicular (annulus) positions.

The first frame and/or second frame may be structured to be inserted into a catheter of a certain size. The frames may be collapsed or compressed from its preformed shape to a size much smaller so as to fit into a catheter. The catheter along with the valve may then be inserted into a cardiac cavity, and upon proper positioning of the catheter, the valve maybe advanced into the native valve with a guide wire. Once the valve is seated in the native valve annulus and leaflets, the artificial valve may be dislodged from the catheter and the catheter may be retracted out of the cardiac cavity.

The second frame may be the primary load bearing element of the valve system. Although not shown, the second frame may further include skirt elements that are structured to avoid paravalvular leakage. These skirts may be made of a flexible fabric. These skirts may further include metal struts embedded within. These metal struts may be made of a shape alloy, such as Nitinol.

The device shown in FIGS. 1 through 6 may be either surgically implanted via open heart surgery directly and sutured directly onto the annulus; may be a part of a sutureless stent design; or may be part of stent design that is implanted via percutaneous or transcatheter procedures. If part of a stent design, the collapsed structure may be mounted onto an external self-expanding stent.

In some embodiments, the valve implant device may be an integrated frame device. The valve implant device may include a singular frame. FIGS. 7 through 21 show additional embodiments of implantable artificial implant valve devices.

FIGS. 7 through 11 show embodiments of an implantable artificial implant valve device for a mitral valve. The valve may be modified according to the anatomy of other valves. For example, additional commissure struts and/or leaflets may be added.

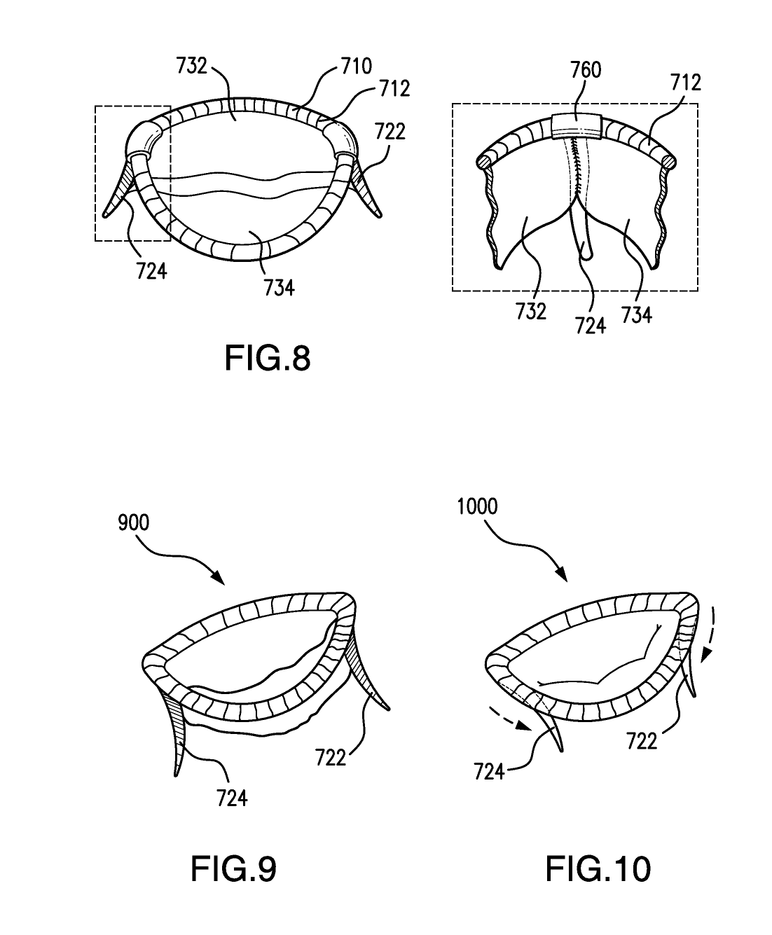

FIGS. 7 and 8 show a side view and a top view, respectively, of an implantable mitral valve device 700. As shown in the side view, the device 700 may include a frame 710. The frame 710 may include a support base (or frame) 712. The base 712 may be an annular ring. The annular ring may have a D-shape to correspond to the anatomy of the mitral valve. The device 700 may further include commissure struts or posts 722 and 724. The number of struts may correspond to the anatomy of the valve. The device 700 may include two posts so as to correspond to the anatomy of a mitral valve. The struts 722 and 724 may extend from the base 712 so as to extend downward into the implanted valve.

The base 712 and the struts 722 and 724 may be made of the same material or of a different material. The base 712 and the struts 722 and 724 may be made of a shape alloy, such as Nitinol.

The device 700 may also include artificial valve 730. The valve may include more than one leaflet. The number of leaflets may depend on the valve anatomy. The valve 730 may be disposed onto the struts 724 and 722. They may be sutured onto the struts. The valve 730 may be made of an artificial polymer or pericardium material. As shown in FIG. 8, the device 700 may have a first (anterior) leaflet 732 and a second (posterior) leaflet 734.

In some embodiments, the leaflets may be secured to each strut. As shown in the enlarged view FIG. 8, the leaflets 732 and 734 may be secured to the strut 724.

In some embodiments, each strut may be may be adjustable. In some embodiments, the frame may include a tension device disposed on each strut that may adjust the configuration of the strut with a complementary device.

In some embodiments, the device 700 may include a tension device 760. The tension device 700 may be a tension device like the device discussed above with respect to the FIGS. 1-6. In some embodiments, the tension device may be structured to adjust the movement of the strut by adjusting the configuration of the strut. In some embodiments, the device may adjust the gap between the leaflets so as to either increase or decrease the movement of the posts. The mechanism (discussed above) may be a zipper-like device.

The operation of the valve device when implanted into a mitral valve is shown in FIGS. 9 and 10. The devices 900 and 1000 include the features of the device 700. The devices FIG. 9 shows the valve device 900 when in a diastolic open position and FIG. 10 shows the valve device 1000 when in a systolic closed position. As shown in these figures, the struts 722 and 724 may be structured to move or swivel. This movement limits the amount of that the leaflets will open. Specifically, the pressure will open the leaflets as much as the struts are structured to swivel or move.

In some embodiments, the valve implant device may be structured to be loaded onto a catheter. Any of the valve implant devices discussed herein may be structured to be loaded onto a catheter. For example, the valve implant devices may be made of a shape alloy, such as Nitinol. In other embodiments, any of the valve implant devices discussed herein may further include a skirt element to avoid paravalvular leakage. The skirt elements may be made of a flexible cloth or material. This cloth or material may be any material that allows tissue growth, such as Dacron. In further embodiments, the skirt elements may include a structure that can adjust the shape of the skirt elements. In some embodiments, this structure may be metal struts made of shape alloy, such as Nitinol. These struts may be inserted behind the leaflets. These struts may be adjusted to thereby adjust the shape of the skirt elements. The adjustment may be based on the anatomy of the patient's valve. In further embodiments, the valve device may further include protrusions that extend from the support frame so as to hold onto the surrounding tissue. These protrusions may be hooks.

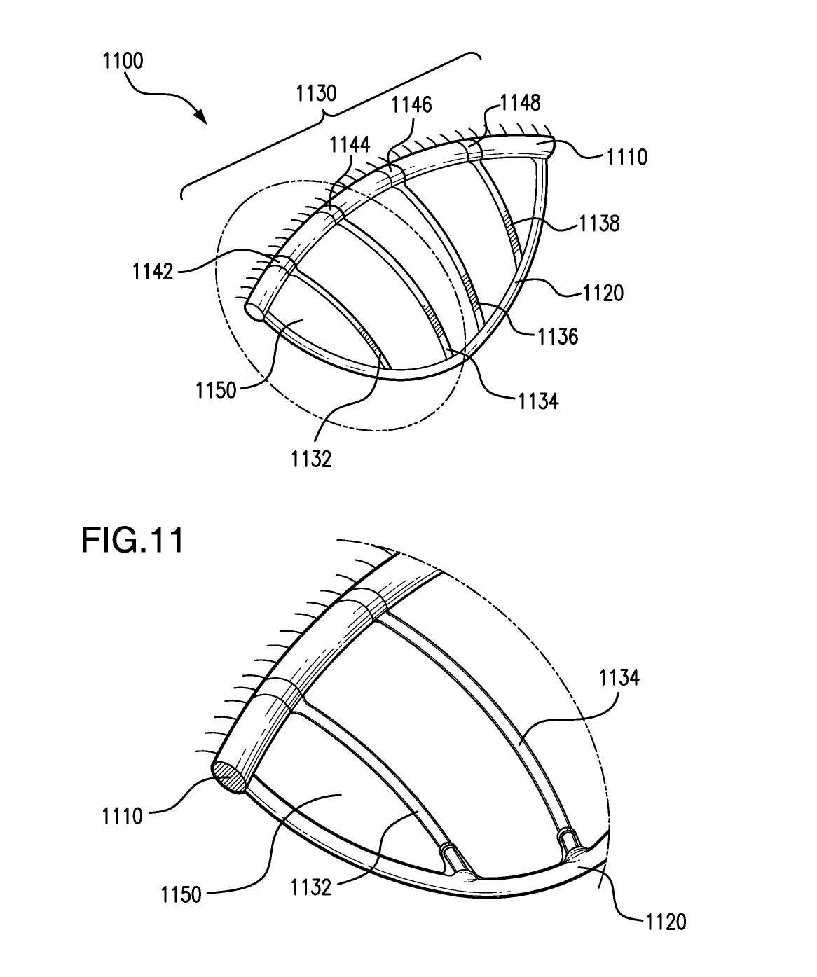

FIGS. 11 through 21 show artificial implant valve devices according to other embodiments. In some embodiments, the valve device may include a frame. The frame may include a support base or frame (later referred to as support frame). The support frame may be an annular ring. The support frame may be made of a rigid material including but not limited to a rigid biocompatible metal, such as stainless steel.

In some embodiments, the device may further include a flexible wire. The flexible wire may be attached to the support base. The flexible wire may be of a flexible material, including but not limited to a memory shape alloy, such as Nitinol. The frame may include the flexible wire.

The device may further include a plurality of flexible elements that extend between and are attached to the support frame and the flexible wire. The flexible elements may be attached to the annular ring and the flexible wire by any known fastener, such as an adhesive or a mechanical fastener. In some embodiments, the device may further include a material that covers the flexible elements and that extends between the flexible wire and the annular ring to form leaflets. The material may be an artificial polymer or pericardium material. The combination of the polymer and the flexible elements may form the structure of the artificial leaflets. The flexible elements may be made may be of a flexible material including, but not limited to, a memory shape alloy such as Nitinol.

In some embodiments, each flexible element may include a connector. The connector may be structured to fixedly dispose each flexible element to the support frame. The connector may be a hook. The support frame may further include a complimentary connector, for example, and indentation or groove.

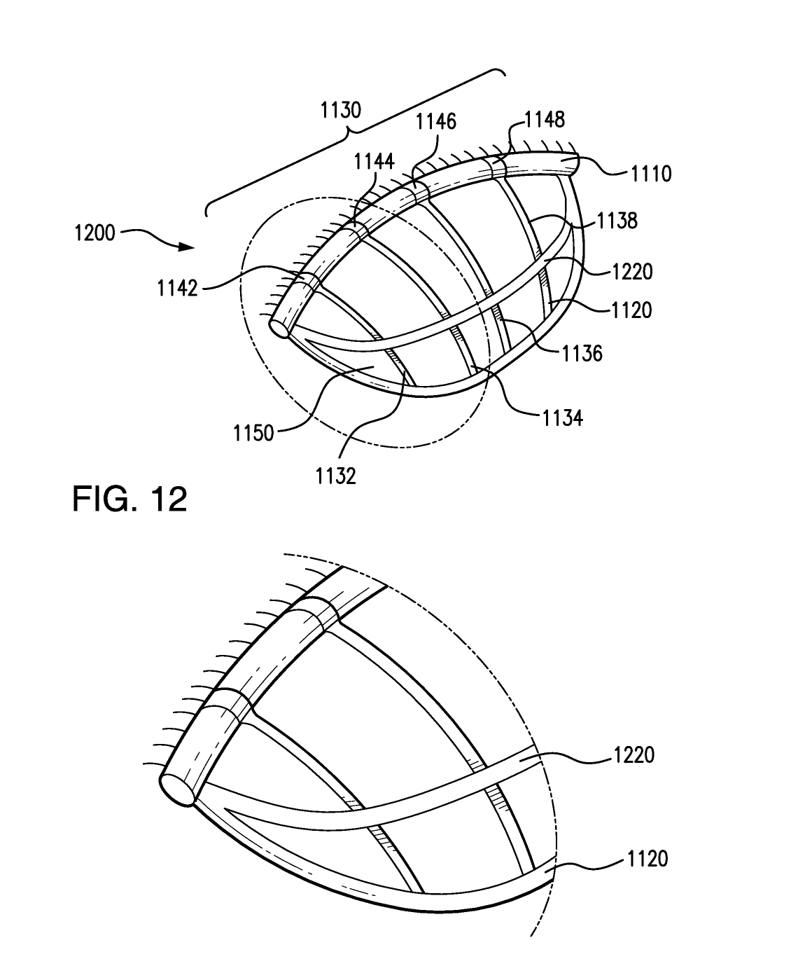

FIGS. 11 and 12 show implant devices 1100 and 1200 according to embodiments. In some embodiments, the devices 1100 and 1200 may include a frame. The devices may include support frame (or support base) 1110. The frame may include the support frame. The support frame may be an annular ring. The devices may further include a flexible wire 1120, and a plurality of flexible elements 1130 which extend between and are attached to the support frame 1110 and the flexible wire 1120. The devices 1100 and 1200 may include four flexible elements 1132, 1134, 1136 and 1138. However, the flexible elements are not limited to four as shown, and the device may include any number of flexible elements, such two, three, five, etc. In some embodiments, the flexible elements may be evenly spaced. In other embodiments, the flexible elements may not be spaced evenly. The elements may be spaced according to anatomy of the valve, such as the shape of the annulus and leaflets. As shown in FIGS. 11 and 12, each of the flexible elements may include a connector. For example, the flexible element 1132 may include a connector 1142, the flexible element 1134 may include a connector 1144, the flexible element 1136 may include a connector 1146, and the flexible element 1138 may include a connector 1148.

In some embodiments, the device may be structured or adjusted according to the anatomy of the valve, such as the annulus and the leaflets. The device may be structured according to the curvature of the native leaflets. In some embodiments, the curvature of the flexible wire may be structured according to the valve leaflets.

In some embodiments, the device may have an even curvature of the flexible elements. The device 1100 shown in FIG. 11 has even curvature. In alternative embodiments, the device may have an uneven curvature. In some embodiments, the uneven curvature may be accomplished by adding additional wires between the wire (1120) and the base support. In some embodiments, the device may include a plurality of wires that are parallel to the base support. For example, the device may include two, three, four, and more flexible wires.

As shown in FIG. 12, the device may include two flexible wires, a first wire and a second wire. The device 1200 may include a second wire 1220 between the (first) wire 1120 and the frame 1110. In some embodiments, the first and second wires may improve the coaptation of the leaflets by providing an area for the coaptation. The area between the first and second flexible wires may be structured for coaptation of the leaflets.

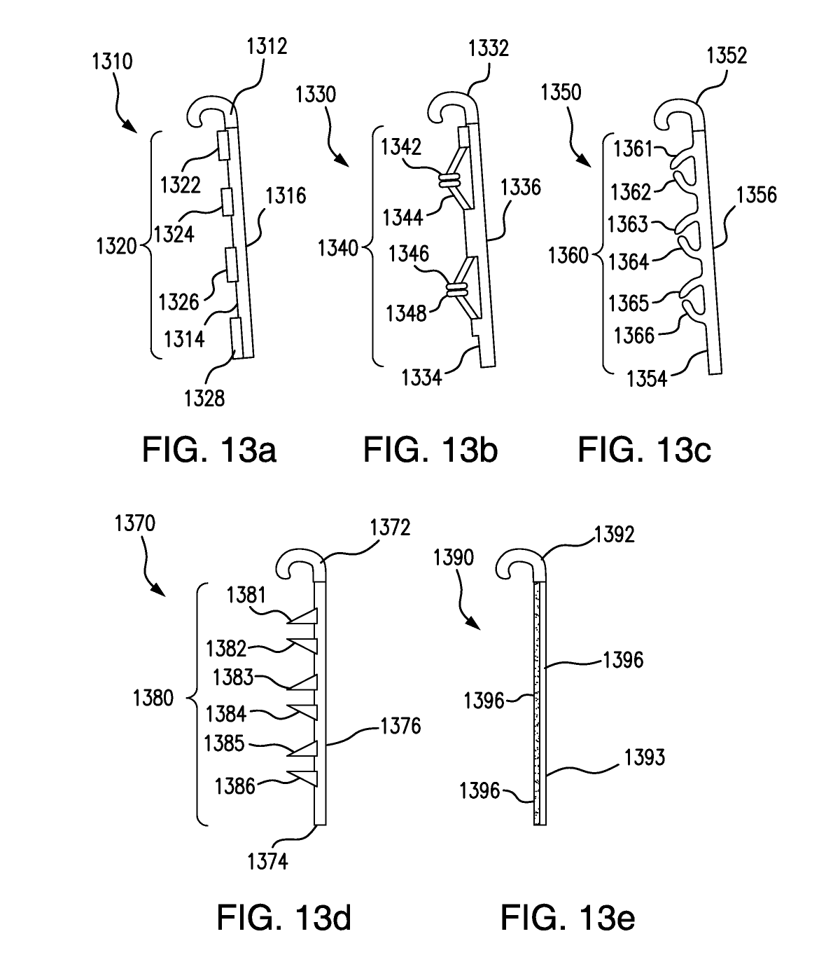

In some embodiments, the flexible elements may be structured or adjusted according to the anatomy of the valve. In some embodiments, the thickness and dimensions of the flexible elements may be chosen based on the anatomy of the valve. In further embodiments, the design of the flexible elements may be chosen based on the desired curvature. FIGS. 13 through 15 show examples of designs for the flexible elements.

In some embodiments, the flexible elements may include a plurality on protrusions on a side. The side may be the left side (the side facing the valve annulus). The protrusions may on the same side of the flexible element as the connector. These protrusions may control the amount of curvature of the flexible element, for example, as shown in FIGS. 15(a) through (d). The protrusions may be structured or adjusted according to the anatomy of the valve, including but not limited to the desired curvature. In some embodiments, the number, the length, and the spacing of the flexible elements may be configured according to the desired curvature. The curvature may be determined based on a scan of the native valve.

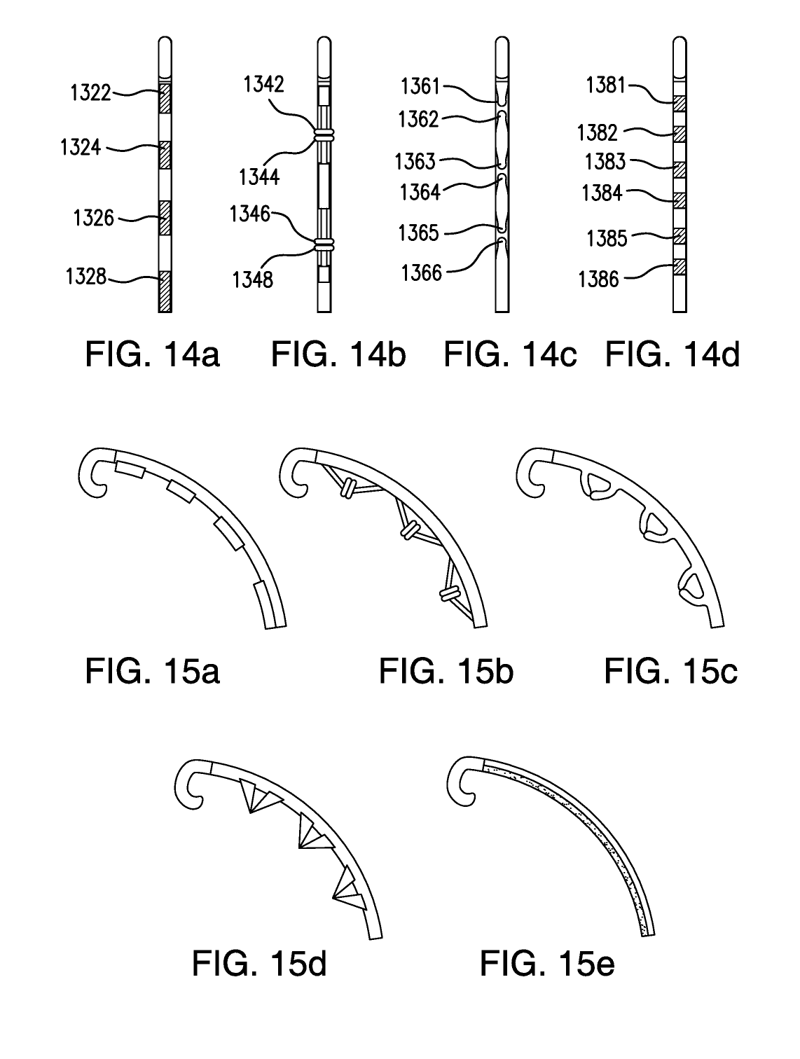

FIGS. 13(a) through (d) show examples of the flexible elements in an unbent state, FIGS. 14(a) through (d) show the respective cross-sectional view, and FIGS. 15(a) through (d) show the respective flexible elements in a bent state. It will be understood that the protrusions are not limited to the designs shown. The protrusions may be of any shape and configuration.

FIGS. 13(a), 14(a), and 15(a) show a flexible element 1310. The flexible element 1310 may include a plurality of protrusions 1320 that are disposed on an outer surface 1314. The flexible element 1310 may include an opposing inner surface 1316. The flexible element may include a connector 1312 that curves toward the outer surface 1314. The protrusions may have a rectangular shape. The flexible element 1310 may include protrusions 1322, 1324, 1326, and 1328. The flexible element may also include more or less protrusions

In some embodiments, the protrusions may be disposed in pairs. Each pair may include protrusions that protrude in opposing directions. The protrusions of each pair may have the same or different shape. The pairs may also be different.

FIGS. 13(b), 14(b), and 15(b) show a flexible element 1330. The flexible element 1330 may include a plurality of protrusions 1340 that are disposed on an outer surface 1334. The protrusions may be organized in pairs, each protrusion of the pair extending in opposing directions. The flexible element 1330 may include an opposing inner surface 1336. The flexible element 1330 may include a connector 1332 that curves toward the outer surface 1334. The flexible element 1330 may include two pairs of protrusions, a first pair of protrusions 1342 and 1344 and a second pair of protrusions 1346 and 1348, that extend in opposing directions. The flexible element may also include more or less pairs.

The pairs of opposing protrusions are not limited to the shape shown in FIGS. 13(b), 13(c), and 13(d). The protrusions may have a different shape. FIGS. 13(c) and (d), 14(c) and (d), and 15(c) and (d), respectively, show examples of different shapes. As shown in FIGS. 13(c), 14(c), and 15(c), a flexible element 1350 may include a plurality of protrusions 1360 having a curved shape disposed on an outer surface 1354. The flexible element 1530 may include an opposing inner surface 1536. The flexible element 1330 may include a connector 1352 that curves toward the outer surface 1354. The flexible element 1350 may include three pairs of protrusions, a first pair of protrusions 1361 and 1362, a second pair of protrusions 1363 and 1364, a third pair of protrusions 1365 and 1366, that extend in opposing directions. The flexible element may also include more or less pairs.

As shown in FIGS. 13(d), 14(d), and 14(d), a flexible element 1370 may include a plurality of protrusions 1380 having an angular shape provided that are disposed on an outer surface 1374. The flexible element 1370 may include a connector 1372 that curves toward the outer surface 1374. The flexible element 1370 may include three pairs of protrusions, a first pair of protrusions 1381 and 1382, a second pair of protrusions 1383 and 1384, a third pair of protrusions 1385 and 1386, that extend in opposing directions. The flexible element may also include more or less pairs.

In other embodiments, the flexible members may include two different materials to control the amount of curvature. The flexible elements may be made of two different materials. The stiffer material may be on the left (the side facing the valve annulus) and a more flexible material may be on the right. The differences between the materials may be used to determine the bending moment. In some embodiments, the bending moment may be determined by the standard bending moment equation.

FIGS. 13(e) and 15(e) show a flexible element 1390 in an unbent state and in a bent state, respectively. The flexible element 1390 may include an inner surface 1393 and an outer surface 1391. The flexible element 1390 may include a connector 1392 that curves toward the outer surface 1391. The flexible element 1390 may include a first material 1394 and a second material 1396. The first material 1394 may be disposed on the outer surface 1391. The second material 1396 may be disposed on the inner surface 1393. The first material 1394 may be stiffer than the second material 1396.

In some embodiments, the flexible elements may be of the same design. In other embodiments, the flexible elements may include a combination of different designs as well as configurations of the design (e.g., the number and length of the protrusions). There may any combination of flexible elements.

In operation, the device may be structured so that the frame is sutured onto the valve annulus and the flexible elements along with the flexible wire(s) protrude into the valve. For example, when implanted into a mitral valve or a tricuspid valve, the left side of the flexible elements having either protrusions or a stiffer material may face the ventricular side of the heart, and the opposing right side may face the atrial side of the heart. The device may be in the opposite positioning for the aortic or pulmonary valve.

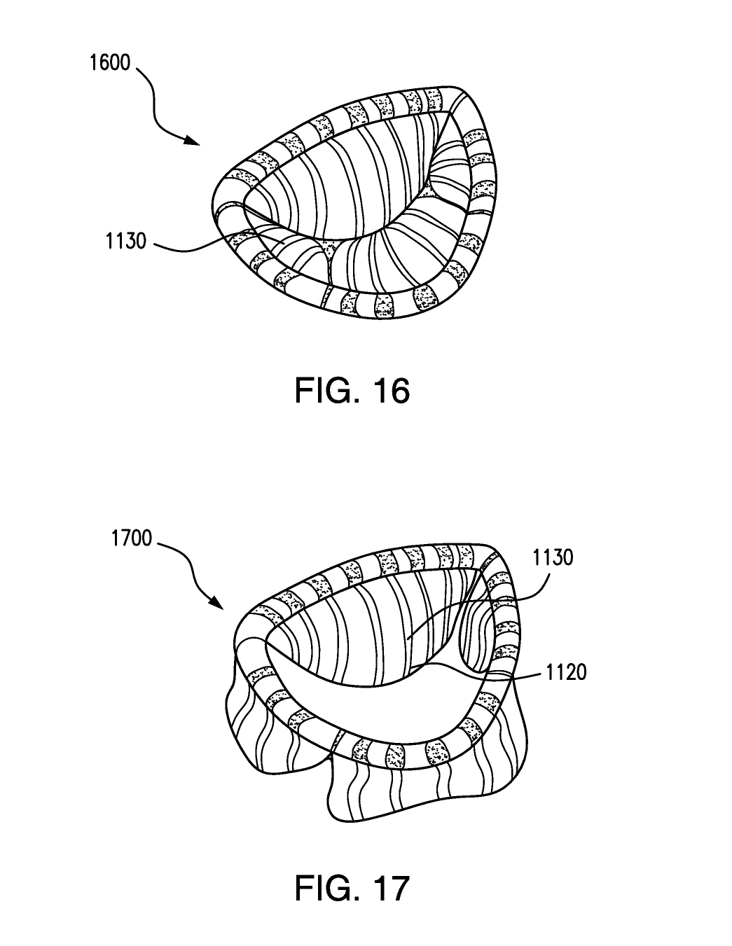

FIGS. 16 and 17 show examples of devices 1600 and 1700 implanted into a valve. The devices 1600 and 1700 include the features of the device 1100. For example, FIG. 16 shows the device 1600 where there is coaptation of the flexible elements (i.e., the artificial leaflets). FIG. 17 shows the device 1700 where the artificial leaflets are open. As shown in these Figures, the flexible elements 1130 may be structured to restrict the movement of the artificial leaflets by restricting the amount of curvature. The flexible wire may also be structured to restrict the movement of the artificial leaflets by further restricting the motion of the flexible elements.

In some embodiments, the device may further include struts that extend from the support frame. In some embodiments, the number of struts may be based on the anatomy of the valve and/or the number of leaflets (native or artificial). The struts may be between each leaflet. The struts may be made of the same material or different material from the support frame and/or the flexible wire.

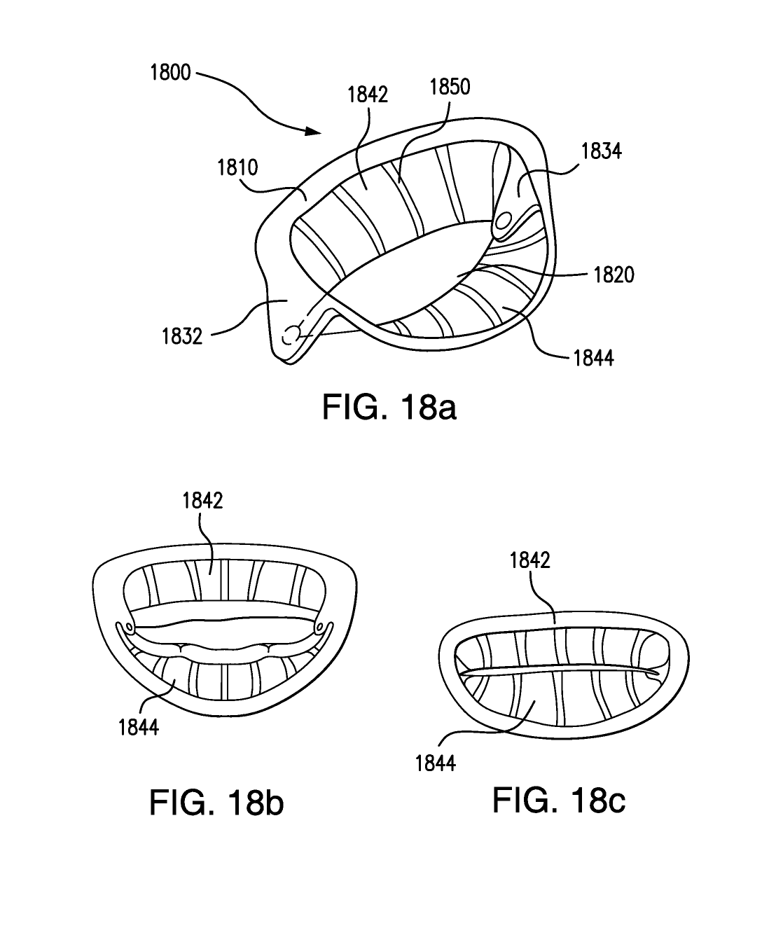

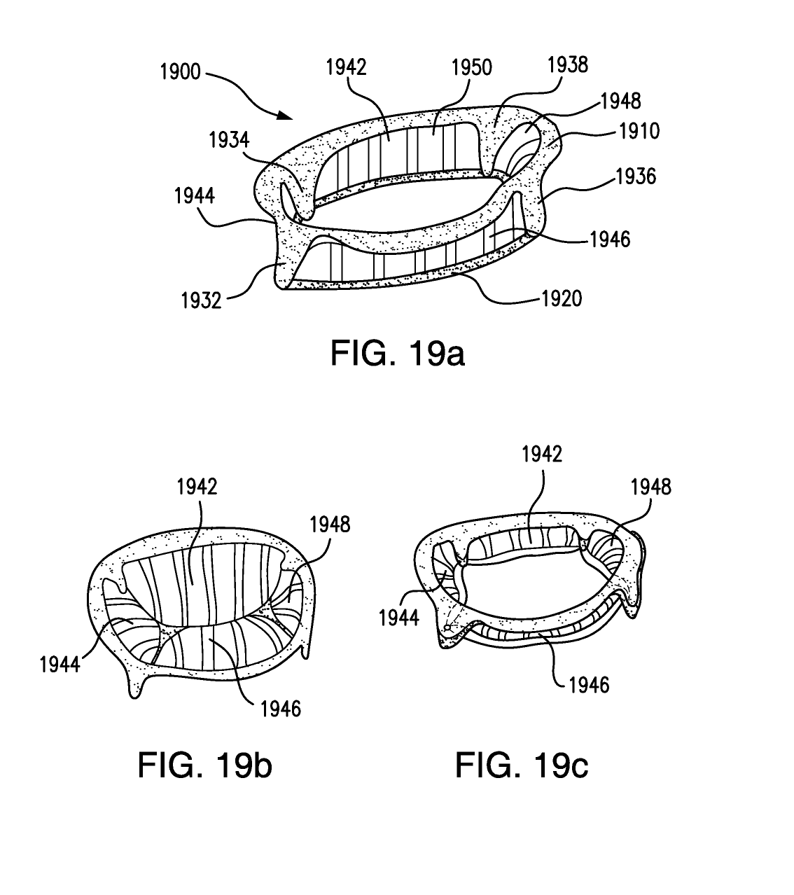

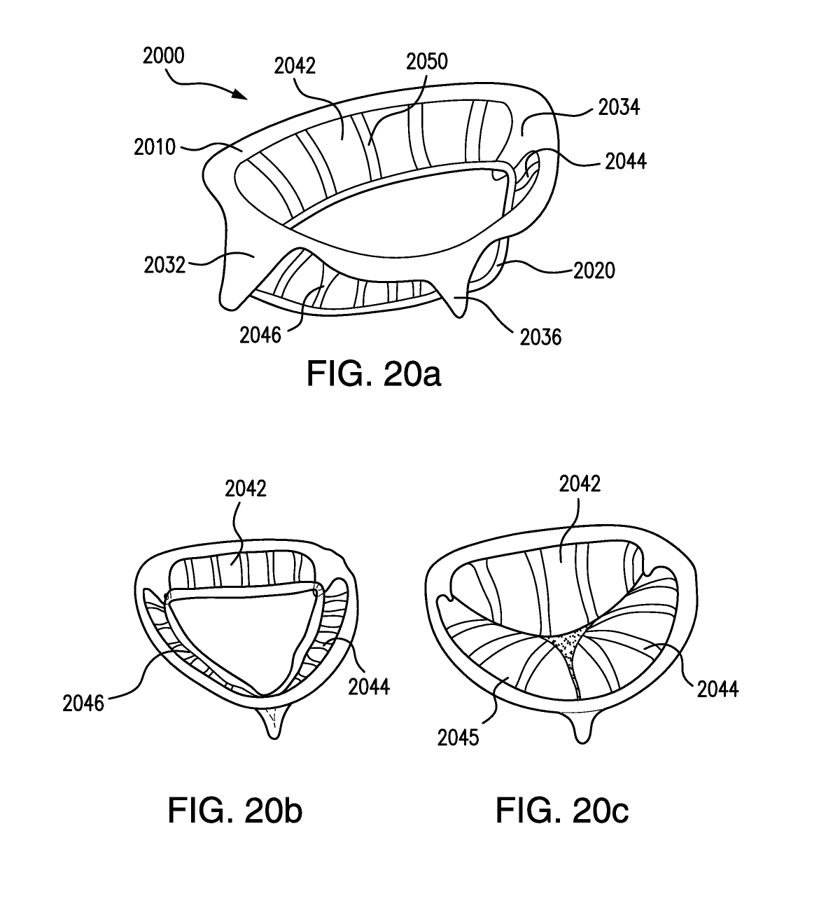

FIGS. 18 through 21 show different designs of the devices that may further include struts. For example, as shown in FIGS. 18(a)-18(c), a device 1800 may include a support frame 1810, a flexible wire 1820, two struts 1832 and 1834, a plurality of flexible elements 1850, and two artificial leaflets 1842 and 1844; as shown in FIGS. 19(a)-19(c), a device 1900 may include a support frame 1910, a flexible wire 1920, four struts 1932, 1934, 1936, and 1938, a plurality of flexible elements 1950, and four artificial leaflets 1942, 1944, 1946, and 1948; and as shown in FIGS. 20(a)-20(c), a device 2000 may include a support frame 2010, a flexible wire 2020, three struts 2032, 2034, and 2036, a plurality of flexible elements 2050, and three artificial leaflets 2042, 2044, and 2046. In some embodiments, the stent may be structured to correspond to the height of the native leaflets. For example, the coapt surface and the flexible members may be structured to correspond to the height.

In operation, in some embodiments, the device may be structured to be sutured to the native valve. The support frame may be sutured to the annulus and the flexible elements may be sutured to the native chordae of the native leaflets. For example, the flexible elements may be sutured directly to the native chordae. In this embodiment, the device may not include a polymer material between the flexible elements. In other embodiments, the device may be used when the native chordae are cut. FIG. 21 illustrates an example of a device 2110 implanted in a valve 2120 when the native chordae are cut. In operation, the base (annular ring) may be sutured to the annulus. In this embodiment, the stent may include a polymer material between the flexible elements to form artificial leaflets.

The valves may be used with a stent and a valve holder, including those discussed below. The valve devices may include any of the alternative embodiments discussed with respect to FIGS. 1-21.

### Stent Systems & Devices

Stent devices (also referred to as “stents”) may be used with or without the valve system according to the disclosure. The stents may be used with different or alternative bioprosthetic valves.

According to embodiments, the stent devices and systems may be used with a replacement valve with or without the valve devices and systems according to the disclosure. The stent devices and systems may be used with different or alternative bio-prosthetic valves.

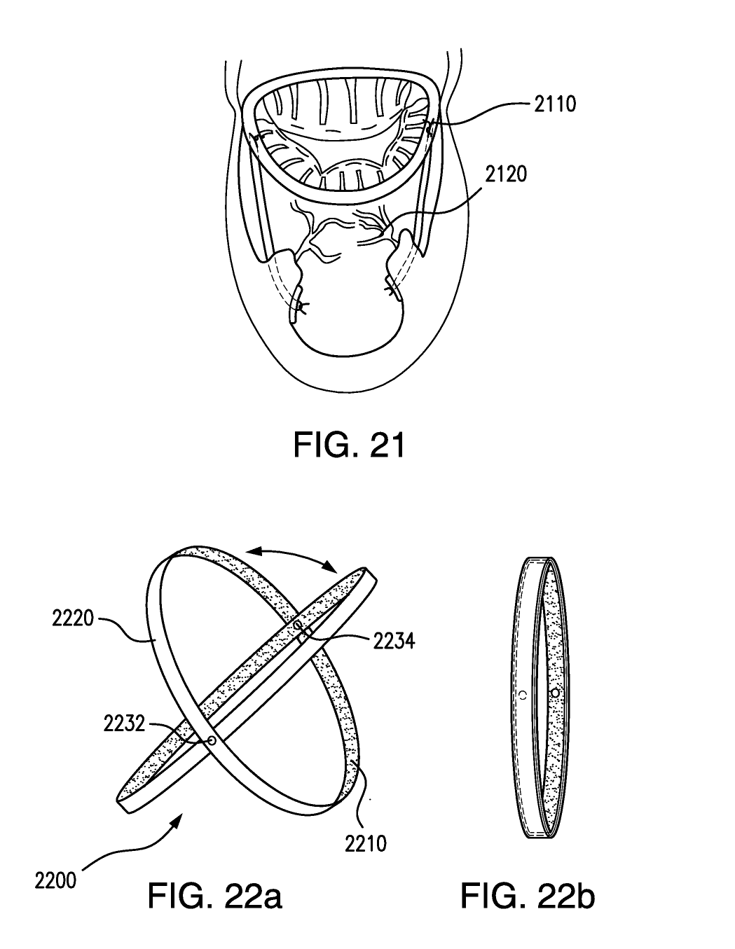

In some embodiments, the stent device may be structured to self-expand and collapse. The stents may also be used to impose a radial force on the native valve annulus and the surrounding valve leaflets. Additionally, because each patient has a different valve anatomy, problems may arise from irregular anatomies causing failure of the valve replacement. Stents according to the disclosure will keep the artificial valve in place irrespective of the shape of the valve. FIGS. 22 through 30 show examples of a stent device according to embodiments.

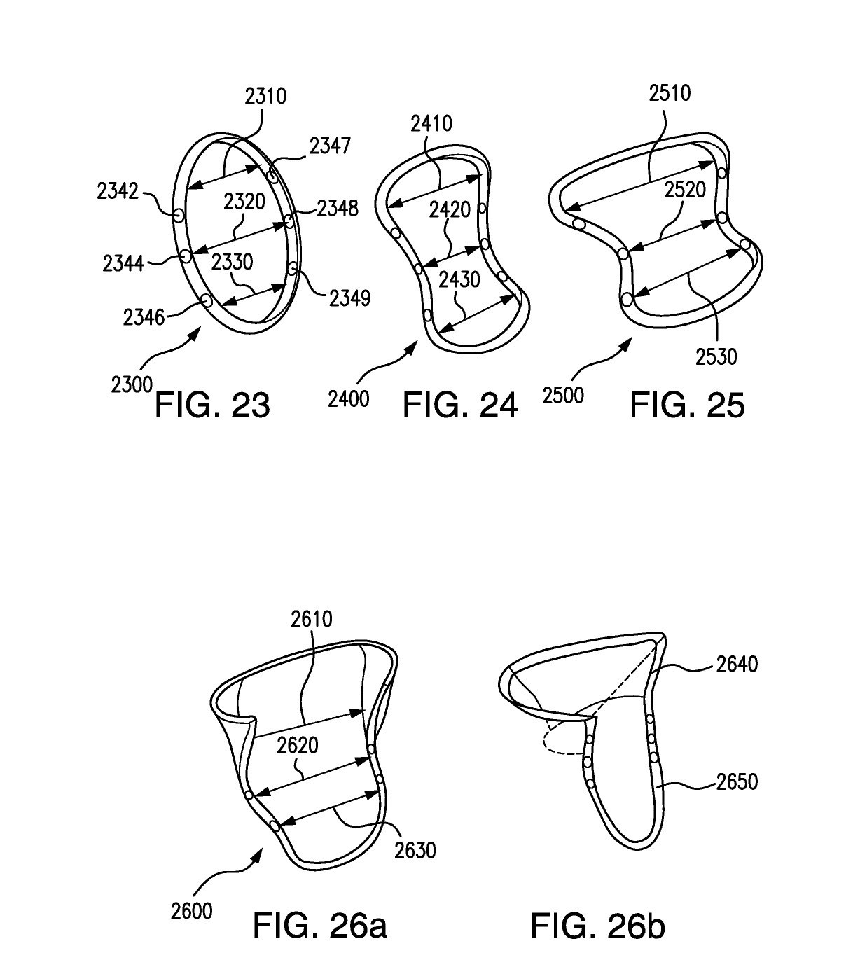

In some embodiments, the stent may include two or more strut segments. For example, the stent may include three, four, or more than four segments. Each strut segment may be partly or completely hollow. The segments may have a ring-like shape. The segments may be of any ring-like shape. For example, the segments have a circular, symmetric, asymmetric, angular, oblong, and oval shape. The segments may be made of a memory shape alloy, such as Nitinol, or a rigid metal. FIGS. 23 through 27 show examples of individual segments.

As shown in FIG. 23, a segment 2300 may have an oval shape. The diameter 2310 may equal the diameter 2330. The diameter 2320, in the center, may be greater than diameters 2310 and 2330.

In some embodiments, the segment may have a symmetric shape. As shown in FIG. 24, a segment 2400 may have a diameter 2410 that is equal to diameter 2430. The diameter 2420, in the center, may be greater than the diameters 2410 and 2430.

In some embodiments, the segment may have an asymmetric shape. As shown in FIG. 25, a segment 2500 may have a diameter 2510 that is greater than diameter 2530. The diameter 2520, in the center, may be less than the diameters 2520 and 2520.

In some embodiments, the segment may be have an oblong shape and may be in two different planes. As shown in FIGS. 26 (a) and (b), a segment 2600 may have a diameter 2610 that is greater than 2630. The diameter 2620, in the center, may be less than the diameters 2610 and 2630. As shown in the side view, FIG. 26(b), the segment 2600 may include a first portion 2640 that protrudes from a second portion 2650. The first portion may be structured to be disposed closer to the atrial, and the second portion may be structured to be disposed closer to the ventricular.

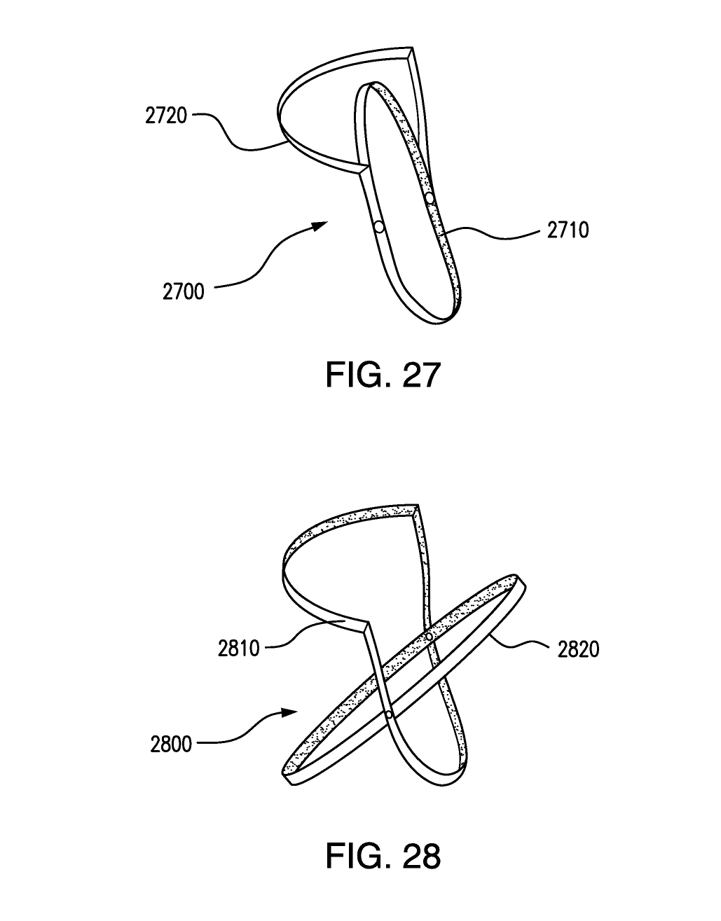

In some embodiments, the segment may include a plurality of struts. As shown in FIG. 27, the segment 2700 may include a circular strut 2710 from which a second strut 2720 may protrude. The second strut 2720 may be circular and extend in perpendicularly from the strut 2710.

The stent may further include at least one connector structured to connect the segments. In some embodiments, the stent may include two connectors to connect the segments at two opposing points. The connectors may be parallel to each other. The connectors may act as a fulcrum about which segments may rotate. The segments may be structured to collapse and expand. In some embodiments, the stent may include more than two connectors.

In some embodiments, each segment may include two openings, one opening for a connector on each side. In other embodiments, the segment may include a plurality of openings on two opposing sides. Each opening may be structured for a connector. In some embodiments, the segment may include six openings, three openings on each side, for example, as shown in the figures. As shown in FIG. 23, the segment 2300 may include three pairs of openings, 2342, 2344, 2346, 2347, 2348, and 2349. In other embodiments, the segment may include more or less openings.

In some embodiments, the segment may include even number of openings. In other embodiments, the segment may include an odd number of openings.

The segments may be structured such that in the expanded state, the stent radially forces against the surrounding tissue. The stent may thus be used in patients with stenosis, such as mitral stenosis. Accordingly, the stents may be implanted without sutures.

In some embodiments, the stent may include segments of the same shape and size. For example, as shown in FIGS. 22(a) and (b), a stent 2200 may include two circular segments 2210 and 2220 and two connectors 2232 and 2234 that connect the two segments at two opposing points. The connections points may be structured to be a fulcrum about which the segments may rotate or pivot. FIG. 22(a) shows the stent 2200 in an open position and FIG. 22(b) shows the stent in a closed and collapsed position.

In other embodiments, the stent may include segments of a different shape and/or size. For example, as shown in FIG. 28, a stent 2800 may include a segment 2810 that is connected to a segment 2820. The segment 2810 has an oblong shape and the segment 2820 has a circular shape. The stent may include any combination of segments and is not limited to the combinations shown in the figures.

In some embodiments, the stent may be structured so that the shape of the frame when in an expanded state may be adjustable. In some embodiments, the stent may be structured for a plurality of configurations. The number of configurations may depend on the number of set of openings provided on each segment. For example, the figures show that each segment may have three sets of openings and thus the stent may be structured for three different configurations. However, each segment may include more or less sets of openings and may be structured for more or less different configurations. The shape of the frame may be adjustable by moving the connector to a different opening.

For example, with three sets of openings, each set being disposed at a different position along the circumference of the segment, the stent may be structured to have a configuration in which the top opening (opening above the connector) is smaller than the bottom opening (opening below the connector); a configuration in which the top and bottom openings are equal; and a configuration in which the top opening is bigger than the bottom opening.

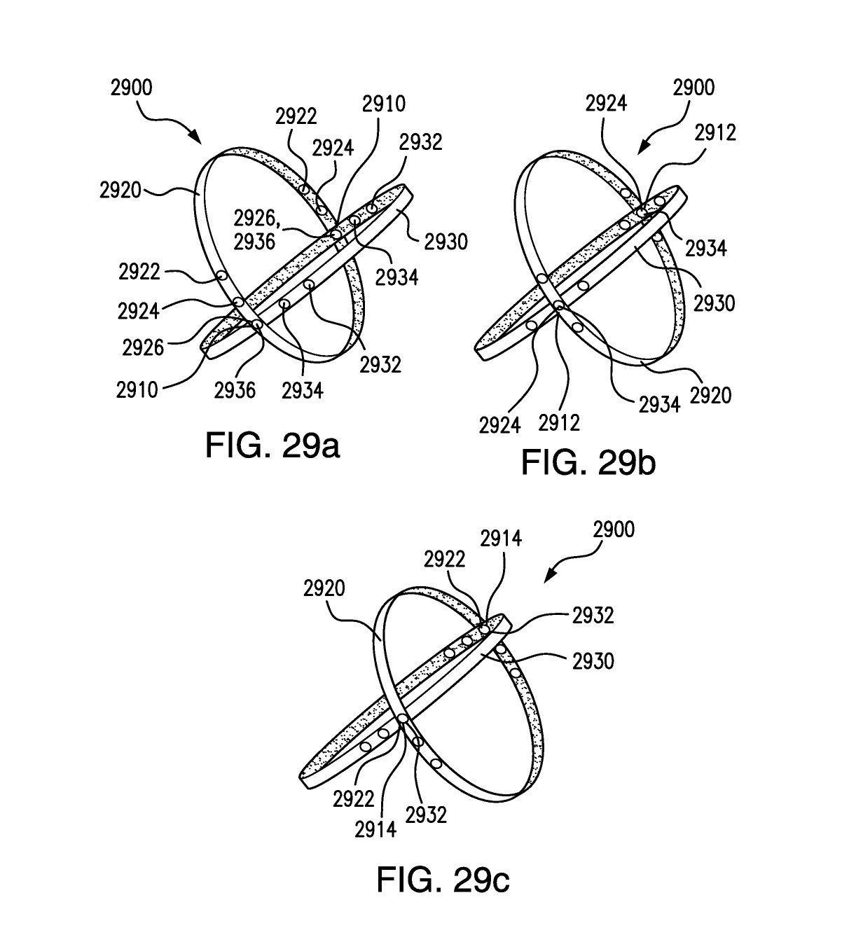

Examples of a stent adjusted to each the three different configurations are shown in FIGS. 29(a)-(c). As shown in FIG. 29, a stent 2900 may include segments 2920 and 2930, of the same circular shape. The segments may be connected by two connectors 2910 at the lowest set (pair) of openings 2926 and 2936. This stent may be structured to have a larger opening on top with a smaller opening at the bottom. This stent may be adjusted so that the top and bottom openings are equal. For example, as shown in FIG. 29(b), the stent 2900 may be adjusted so that the segments 2920 and 2930 are connected at the center set of opening 2924 and 2934 by two connectors 2912. The stent may be further adjusted so that the top opening is bigger than the bottom opening. As shown in FIG. 29(c), the stent 2900 may be further adjusted so that the segments 2920 and 2930 are connected at the highest set of openings 2922 and 2932 by two connector 2914.

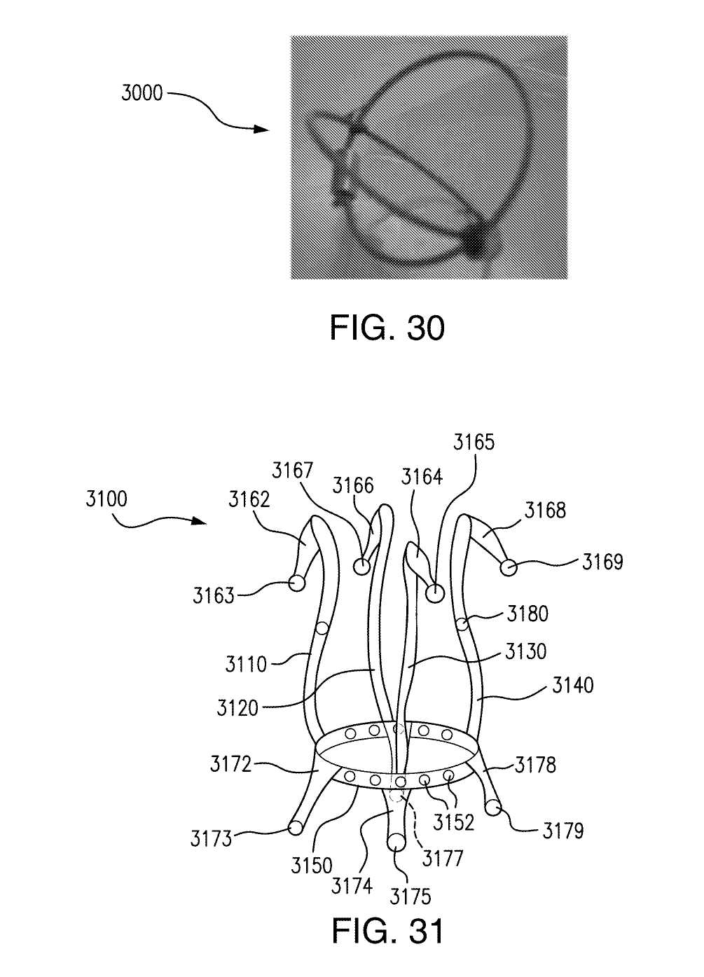

FIG. 30 shows an example of a prototype of a stent device 3000 according to some embodiments.

In some embodiments, stent devices and/or implant devices according to embodiments may be used with a valve retainer device. The valve retainer may fixedly dispose an artificial valve or implant device for implantation.

In some embodiments, the valve retainer device may include a frame. The frame may be circular frame. The frame may include a plurality of openings along the circumference for sutures. The openings maybe structured to secure an artificial valve to the valve retaining device with sutures.

The valve retaining device may include a plurality of extending members. The extending members may extend from the frame. The retaining device may include two, three, four, or more extending members. The extending members may be structured to form an opening. The number, shape, and configuration of the extending members may be based on the valve device to be implanted, as well as the valve anatomy. The connector may be an opening structured to receive sutures, or may be a groove or indentation.

In some embodiments, each of the extending members may include (top) struts that extend therefrom in an opposing direction. In some embodiments, the frame may also include (leg) struts that extend in the same direction as the top struts. The struts may be structured to secure the valve retaining device to a stent device. One, some, or all of the struts may include a bolus at an open structured to fixedly connect the valve retaining device to a stent device. The struts may extend over or overhang a top edge and/or the bottom edge when placed within a stent device. In some embodiments, one or all of the extending members may include a connector for the artificial valve.

The valve retainer device may be made of a member shape alloy, such as Nitinol, or a more rigid biocompatible metal.

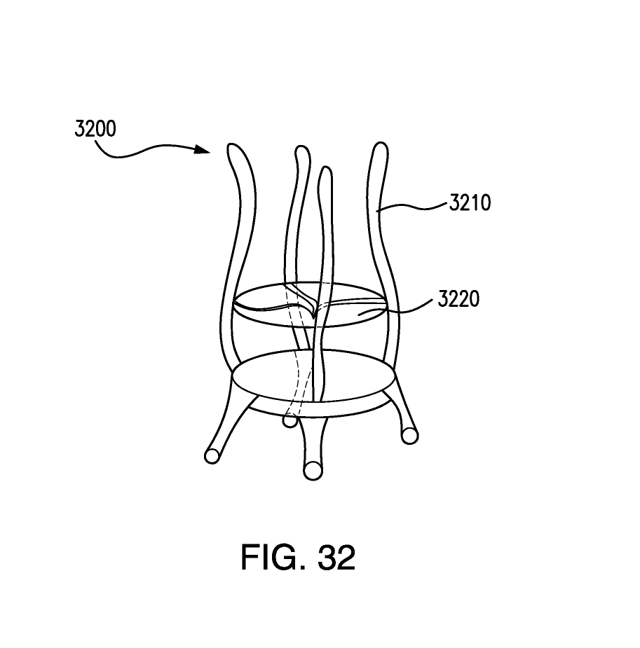

FIGS. 31 and 32 show examples of a valve retainer device. As shown in FIG. 31, a valve retainer device 3100 may include a circular frame 3150 from which a plurality of extending members are attached. The valve retainer device may include any number of extending members and is not limited to the four shown in the figure. The frame 3150 may include a plurality of openings 3152 structured for sutures. The valve retainer device 3150 may include four extending member 3110, 3120, 3130, and 3140. Each of the extending members may include top struts 3162, 3164, 3166, and 3168 respectively that extend away from, in an opposing direction, from the extending members. The struts may each include bolus 3163, 3165, 3167, and 3169. The retainer device may further include bottom struts 3172, 3174, 3176 (not shown), and 3178 that extend from the frame 3150 in the same direction as the top struts. The bottom struts may each include bolus 3173, 3175, 3177, and 3179. The device 3100 may include a connector 3180 on each extending members to position the valve.

FIG. 32 shows an example of an artificial valve implant device 3220 secured to a retainer device 3210. The device may be structured for the pericardial valve.

### Kits

According to some embodiments, one, some or all components of the devices and system may be structured for single use or be disposable. In some embodiments, one, some or all components may be sterilized. According to some embodiments, a portion or combination of the single use items may be sold as kit.

In some embodiments, the kit may include an implant device according to embodiments. The kit may include at least one frame. In some embodiments, the kit may include different flexible members. In some embodiments, the kit may include a stent device. In some embodiments, the kit may include a plurality of different segments. In some embodiments, the kit may include one, some, or all of an implant device, a stent and a valve retainer device. In some embodiments, the kit may include a valve implant system that may include a stent and an implant device.

While various embodiments of the disclosure have been described, the description is intended to be exemplary rather than limiting and it will be appeared to those of ordinary skill in the art that may more embodiments and implementations are possible that are within the scope of the disclosure. Those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed is:

1. A valve implant device for surgical replacement of a valve of a patient, the valve having a valve annulus, comprising:
a frame structured to have a shape of the valve to be replaced and being structured for placement within the valve annulus,
at least one valve leaflet, the leaflet including artificial chordae tendineae that is structured to control the motion of the valve beyond the valve annulus.

2. The device according to claim 1, wherein the device includes more than one leaflet.

3. The device according to claim 2, wherein the leaflets are the same size.

4. The device according to claim 2, wherein the leaflets are of different sizes.

5. The device according to claim 1, wherein:
the frame includes a support base and two or more struts that extend from the structure perpendicularly from a fulcrum,
the struts are structured to have adjustable flexibility, and
the struts are structured to be adjusted either before or after surgical implantation to alter the motion of the valve leaflets.

6. The device according to claim 5, wherein the valve leaflet includes a material mounted on the frame, and the material is an artificial polymeric material or treated tissue.

7. The device according to claim 1, wherein the frame includes a support structure, the device further comprising:
two or more struts that extend perpendicularly from the frame; and
a connector disposed on each strut;
wherein each strut includes at least one tension member that extends from the support frame to the connector.

8. The device according to claim 7, wherein the frame includes a tension device that is structured to adjust a length and a tension of the member.

9. The device according to claim 8, wherein the length and the tension is adjusted according to at least one of desired extension of leaflet motion and recoil force to retract the valve leaflet.

10. The device according claim 7, wherein:
the device includes a first frame and a second frame, the frame being structured for placement within the valve annulus being the first frame, the second frame including the at least one valve leaflet;
the second frame includes a support base and at least two struts that extend perpendicularly from the support base at a fulcrum, wherein the struts are structured to be flexible bend with the fulcrum.

11. The device according to claim 9, wherein the second frame includes at least two connectors, each connector structured to connect with the connector of the first frame.

12. A stent device structured for use with a valve implant device for replacement of a heart valve of a patient, the valve having native valve leaflets and orifice, the stent comprising:
two segments, the two segments structured to receive a valve implant device.

13. The stent according to claim 12, wherein the stent is structured to radially push the native valve leaflets outwards and rest on an annulus of the valve and during ventricular pressurization provide a radially inward force to hold the valve implant device in place.

14. The stent according to claim 12, wherein each segment has a same shape and size.

15. The stent according to claim 12, wherein each segment has a different shape and size.

16. The stent according to claim 12, further comprising:
at least two connectors, each connector connecting the two segments,
wherein the segments are structured to rotate about the connector.

17. The stent device according to claim 16, wherein each segment is structured for a plurality of configurations.

18. An implant system for surgical replacement of a valve of a patient, the valve having a valve annulus, a orifice and native leaflets, comprising:
an implant valve device structured to have a shape of the valve to be replaced and being structured for placement within the valve annulus, the device including at least one frame and at least one valve leaflet, the leaflet including artificial chordae tendineae that is structured to control the motion of the valve beyond the valve annulus; and
a stent device, the stent device being structured to have a shape of the valve so that when a radial force is applied on the native valve leaflets when the stent is implanted in the orifice, a passage is formed in the valve, the passage being structured to receive the implant valve device when implanted into the orifice.

19. The implant system according to claim 18, wherein the stent device includes two movable segments.

20. The implant system according to claim 19, further comprising:
a valve retainer device structured to secure the implant valve device, the valve retainer device being structured to fixedly connect to the stent device.
